(19) United States
(12) Patent Application Publication
(10) Pub. No.: US 2023/0158311 A1
(43) Pub. Date: May 25, 2023

(54) Brain Cardiac Pacemaker

(71) Applicant: Philip Chidi Njemanze, Owerri (NG)

(72) Inventor: Philip Chidi Njemanze, Owerri (NG)

(21) Appl. No.: 17/530,491

(22) Filed: Nov. 19, 2021

(51) Int. Cl. A61N 1/37; A61N 1/375; A61N 1/362; A61N 1/365; A61B 8/06; A61B 8/00

(52) U.S. Cl. CPC A61N 1/3702; A61N 1/37512; A61N 1/3629; A61N 1/36571; A61N 1/36528; A61B 8/06; A61B 8/56; A61B 2560/0209

(57) ABSTRACT

The present invention provides monitoring of brain blood circulation in a patient with cardiac pacemaker for the prevention of symptoms associated with pacemaker syndrome and stroke. The monitoring of brain blood flow velocity is performed using a transcranial Doppler ultrasound device synchronized with an implanted cardiac pacemaker, to select the pacing mode that enhances cerebral perfusion in the patient. The system further detects microembolic signals in the cerebral circulation and triggers sonothrombolysis as well as release of thromolytic and neuroprotective agents for clot dissolution.

### CROSS-REFERENCE TO RELATED APPLICATION

### U.S. Patent Documents

| Document Number | Date | Name | Classification | Cited by |
|---|---|---|---|---|
| 1. U.S. Pat. No. 6,390,979 B1 | May 2002 | Njemanze, Philip C. | 600/438 | Inventor |
| 2. U.S. Pat. No. 6,468,219B1 | October 2002 | Njemanze, Philip C. | 600/454 | Inventor |
| 3. U.S. Pat. No. 6,547,737B2 | April 2003 | Njemanze, Philip C. | 600/454 | Inventor |
| 4. U.S. Pat. No. 6,663,571 | December 2003 | Njemanze Philip C. | 600/504 | Inventor |
| 5. U.S. Pat. No. 6,773,400 | July 2004 | Njemanze Philip C. | 600/454 | Inventor |
| 6. U.S. Pat. No. 7,942,820 | May 2011 | Njemanze Philip C. | 600/441 | Inventor |
| 7. U.S. Pat. No. 8,152,727 | April 2012 | Njemanze Philip C. | 600/454 | Inventor |
| 8. EP 2,471,576A1 | December 2014 | Jacobson Peter M. | A61N1/056 | Inventor |

### OTHER PUBLICATIONS

Alexandrov A V, Demchuk A M, Felberg R A, Christou I, Barber P A, Burgin W S, Malkoff M, Wojner A W, Grotta J C. (2000). High rate of complete recanalization and dramatic clinical recovery during tPA infusion when continuously monitored with 2-MHz transcranial Doppler monitoring. Stroke. 31(3):610-614. doi: 10.1161/01.str.31.3.610. PMID: 10700493.
Awadh M, MacDougall N, Santosh C, Teasdale E, Baird T, Muir K W. (2010). Early Recurrent Ischemic Stroke Complicating Intravenous Thrombolysis for Stroke: Incidence and Association With Atrial Fibrillation. Stroke. 41:1990-1995 https://doi.org/10.1161/STROKEAHA.109.569459
Davis S M, Lees K R, Albers G W, Diener H C, Markabi S, Karlsson G, Norris J. (2000). Selfotel in acute ischemic stroke : possible neurotoxic effects of an NMDA antagonist. Stroke. 31(2):347-54. doi: 10.1161/01.str.31.2.347. PMID: 10657404.
Heiss W D, Thiel A, Grond M, Graf R. (1999). Which targets are relevant for therapy of acute ischemic stroke? Stroke. 30(7):1486-9. doi: 10.1161/01.str.30.7.1486. PMID: 10390327.
Koide H, Kobayashi S, Kitani M, Tsunematsu, Y Nakazawa Y. (1994). Improvement of cerebral blood flow and cognitive function following pacemaker implantation in patients with bradycardia. Gerontology, 40(5), 279-285. doi: 10.1159/000213597.
Lees K R. (1997). Cerestat and other NMDA antagonists in ischemic stroke. Neurology 49; pp. S66-S69
Magjarevic R, Ferek-Petric B. (2010). Implantable Cardiac Pacemakers-50 Years from the First Implantation. Slovenian Medical Journal 79(1):55-67.
Molina, C. A., Barreto, A. D., Tsivgoulis, G., Sierzenski, P., Malkoff, M. D., Rubiera, M., Gonzales, N., Mikulik, R., Pate, G., Ostrem, J. (2014). Transcranial ultrasound in clinical sonothrombolysis (TUCSON) trial, Ann. Neurol. 66 (2009) 28-38.
Njemanze, P C. (1992a). Cerebrovascular dysautoregulation syndrome in a heart-lung transplant recipient. J Cardiovascular Tech, 10, 227-232.
Njemanze, P C. (1992b). Critical limits of pressure flow relation in the human brain. Stroke, 23, 1743-1747.
Njemanze, P C. (1993a). Cerebral circulatory changes in case of pacemaker syndrome. Journal of Cardiovascular Technology, 11, 105-109.
Njemanze, P C. (1993b). Cerebral circulation dysfunction and hemodynamic abnormalities in syncope during upright tilt test. The Canadian Journal of Cardiology, 9 (3), 238-242.
Njemanze, P C. (1993c). Isoproterenol induced cerebral hypoperfusion in a heart transplant recipient. Pacing and Clinical Electrophysiology: PACE, 16 (3 Pt 1), 491-495.
Njemanze, P C. (1994). Cerebrovascular dysautoregulation syndrome complex-brain hypoperfusion precedes hypotension and cardiac asystole. Jpn Cir J. 58, 293-297.
Paulson O B, Strandgaard S, Edvinsson L. (1990). Cerebral autoregulation. Cerebrovasc Brain Metab Rev. 2, 161-192.
Purkayastha, S., & Sorond, F. (2012). Transcranial Doppler ultrasound: technique and application. Seminars in neurology, 32(4), 411-420. https://doi.org/10.1055/s-0032-1331812
Shapiro W, Chawla, N P S.(1969). Observations on the regulation of cerebral blood flow in complete heart block. Circulation, XL, 863-870.
Simon R, Shiraishi K. (1990). N-methyl-D-aspartate antagonist reduces stroke size and regional glucose metabolism. Annals of Neurology, 27; 606-611.
Sulg, I A., Cronqvist, S., Schuller, H., Ingvar, D H. (1969).The effect of intracardial pacemaker therapy on cerebral blood flow and electroencephalogram in patients with complete atrioventricular block. Circulation 36: 487.
Todua F., Gachechiladze D. (2018). Detection of Cerebral Microemboli. In: Noninvasive Radiologic Diagnosis of Extracranial Vascular Pathologies. Springer, Cham. https://doi.org/10.1007/978-3-319-91367-4_21 .
Yang W., Zhou Y. (2017). Effect of pulse repetition frequency of high-intensity focused ultrasound on in vitro thrombolysis, Ultrasonics Sonochemistry, 35; Part A, 152-160.
Zhou Y, R. Ramaswami R. (2014). Comparison of sonothrombolysis efficiencies of different ultrasound systems, J. Stroke Cerebrovasc. Dis. 23; 2730-2735.

### STATEMENT REGARDING FEDERALLY SPONSORED RESEARCH OR DEVELOPMENT

Not applicable

### REFERENCE TO MICROFICHE APPENDIX

Not Applicable

### FIELD OF THE INVENTION

The present invention generally relates to brain and cardiac pacemaker for monitoring of brain blood flow velocity using transcranial Doppler ultrasound during cardiac pacing. Specifically, this invention is a hybrid device that comprises a cardiac pacemaker and an implanted transcranial Doppler ultrasound used to synchronize cardiac pacing with cerebral blood flow measurements and microembolic signal detection for the prevention of symptoms associated with cardiac pacemaker syndrome and stroke.

### BACKGROUND OF THE INVENTION

The conventional cardiac pacemakers are of three different types: (1) Single chamber pacemaker. This type of pacemaker usually carries electrical impulses to the right ventricle of the heart. (2) Dual chamber pacemaker. This type of pacemaker carries electrical impulses to the right ventricle and the right atrium of the heart to help control the timing of contractions between the two chambers. (3) Biventricular pacemaker. This type of pacemaker stimulates both of the lower heart chambers (the right and left ventricles) to make the heart beat more efficiently. Biventricular pacing, also called cardiac resynchronization therapy, is for patients with heart failure and heartbeat problems. In some patients, symptoms may arise that are associated with the cardiac pacemaker implantation, and have been described as pacemaker syndrome. Pacemaker syndrome comprises a constellation of symptoms resulting from loss of atrioventricular synchrony or retrograde ventriculoatrial conduction. There is lack of sequential or physiological atrioventricular filling due to atrial_contraction against closed atrioventricular valves during ventricular systole, transient increases in atrial pressure may result. This may precipitate symptoms including fatigue, exercise intolerance, a sensation of fullness, dyspnea, and headache. It may also provoke vagal mediated changes associated with dizziness and syncope. Pacemaker syndrome in patients is more frequently associated in those implanted with single-chamber ventricular pacing systems, but can also be seen with dual-chamber systems with inappropriate atrioventricular delay settings. Although, it is less common in children than adults, it may develop over time. State-of-the art treatment can include adjustment of atrioventricular intervals (including rate adaptive features) for those with dual-chamber systems and upgrading those with single-chamber devices to a dual-chamber system. However, in some cases even the upgrade to dual-chamber systems does not resolve the symptoms. Transcranial Doppler sonography was used to access cerebral blood flow velocity in a patient with pacemaker syndrome and demonstrated that, the lightheadedness and syncope was associated with diminution of cerebral blood flow velocity and decreased pulsatility (Njemanze, 1993a). The absence of evidence for increased cerebrovascular resistance suggests that, hypotension resulting from vagal induced vasodepressor response is the leading pathophysiologic mechanism in ventricular pacemaker syndrome (Njemanze, 1993a). On the other hand, in some patients, the symptoms of dizziness and syncope were related to sudden diminution in cerebral blood flow without hypotension described as cerebral syncope Type 1 or cerebrovascular dysautoregulation syndrome (Njemanze, 1993a, 1993b). It has been demonstrated in a cardiac transplant patient with total heart de-afferentation, that cardiac reflexes do not have a direct role in causing cerebrovascular dysautoregulation syndrome but rather the mechanisms were linked to raised sympathetic tone (Njemanze 1993c). Furthermore, cardiopulmonary reflexes were not implicated in the pathogenesis of cerebrovascular dysautoregulation syndrome as demonstrated in a heart-lung transplant recipient with total heart and lung de-afferentation (Njemanze 1992a). Moreover, in cerebrovascular dysautoregulation syndrome complex, the brain hypoperfusion preceded hypotension and cardiac asystole (Njemanze, 1994). The changes of brain blood flow observed show that, the symptoms of pre-syncope arise when diminution of cerebral blood flow velocity attained a reduction of between −25% to −50%, and at −50% it reached a critical limit, that does not support pressure-flow relations in the human brain resulting in loss of consciousness or syncope (Njemanze, 1992b). Cerebral blood flow autoregulation is the ability of the brain to maintain relatively constant blood flow despite changes in perfusion pressure (Paulson et al. 1990). There is a direct relationship between heart rate and cerebral blood flow perfusion. The occurrence of reversible mental symptoms in patients with complete heart block and normal arterial blood pressures suggests involvement of underlying inadequate cerebral perfusion. Pacemaker-induced restoration of heart rate has been reported to be accompanied, after varying duration of time, by increases in regional cerebral blood flow and improvement in the electroencephalogram in subjects with complete heart block (Sulg et al. 1969). It has been demonstrated that, in the presence of heart block associated with biventricular failure, both cerebral blood flow and cardiac output are reduced. Furthermore, both cardiac output and cerebral blood flow were significantly increased by pacing at 60 beats per minute; pulmonary, systemic, and cerebrovascular resistances declined pari passu as a result of these increases in flow (Shapiro et al. 1969). It has been demonstrated that, changes in cerebral blood flow significantly correlated with changes in heart rate, but not with changes in cardiac output (Koide et al., 1994). The patients were examined before the pacemaker implantation, and the verbal cognitive function was lower in bradycardic patients than in age-matched control subjects. The brain CT revealed significant advanced atrophy in these patients. However, verbal cognitive function was also improved after the cardiac pacemaker implantation. The investigators concluded that, pacemaker implantation in the severe bradycardic elderly should be beneficial not only for cardiac function but also for brain function. Furthermore, that heart rate is one of the important factors in the regulation of cerebral circulation in patients with severe bradycardia. Even though, clinical observations show that, functional cardiac pacemakers in the elderly improved quality of life and may prevent mental deterioration, conventional devices were not designed to maximize improvements in mental performance. What is desirable is a system that synchronizes cardiac pacemaker function with cerebral perfusion. Moreover, it has been demonstrated that cerebral blood flow velocity could be used to assess the state of mental performance (Njemanze, 2005) and the device described in detail in U.S. Pat. No. 6,390,979 to Njemanze. The present invention assesses the state of mental-being of the patient using the patterns of cerebral blood flow velocity changes according to the teachings of U.S. Pat. No. 6,390,979 to Njemanze, 2002, before and during cardiac pacing to determine changes under different modes of cardiac pacing for the purpose of selecting the best mode of cardiac pacing to maintain best mental performance.

Over 4 million people around the world are living with implanted cardiac devices. The incidence of pacemaker syndrome was 13.8% at 6 months, 16.0% at 1 year, 17.7% at 2 years, 19.0% at 3 years, and 19.7% at 4 years. The incidence of pacemaker syndrome has been estimated to range from 5% (symptoms severe enough to warrant pacemaker revision) to 80% (mild to moderately severe symptoms). In many patients, asymptomatic pacemaker syndrome is probably common and the true incidence of pacemaker syndrome much higher. The growing problem of pacemaker syndrome is due in-part to the lack of synchronization with cerebral blood flow circulation, which is the subject of the present invention. In addition to the problem of pacemaker syndrome, cerebral microembolism can occur in cardiac patients.

Cerebral embolism from arterial and cardiac sources may account for as many as 30-60% of all strokes, the detection of microemboli could be of great importance for the identification of patients with implanted cardiac pacemaker who are at high risk for cerebral embolism. The state-of-the-art pacemakers have no provision for measurement of cerebral blood flow and detection of microemboli in cerebral arteries, which are major determinants of patient's symptoms. The present innovation called Brain Cardiac Pacemaker measures cerebral blood flow velocity indexed by implanted transcranial Doppler ultrasound and detects microemboli in cerebral arteries as well as performs cardiac pacing to maximize improvement in cerebral blood flow. In another embodiment, the present invention functions as an external transcranial Doppler ultrasound device telemetrically linked to the implanted cardiac pacemaker. It is hoped that, the application of Brain Cardiac Pacemaker would greatly improve the clinical outcomes and quality of life for patients with pacemakers.

### BRIEF SUMMARY OF THE INVENTION

There is currently no method to synchronize cardiac pacing with monitoring of cerebral blood flow velocity in real-time. Transcranial Doppler ultrasound is a non-invasive technique with high temporal resolution for measurement of cerebral blood flow velocity which varies proportionally with cerebral blood flow and is used in the clinical setting to monitor cerebral blood perfusion (Njemanze, 1992a, 1992b, 1993a, 1993b, 1993c, 1994, Molina et al, 2009). Other neuroimaging techniques such as positron emission tomography (PET) and functional magnetic resonance imaging (fMRI) and single-photon emission computerized tomography (SPECT) are cumbersome and have poor temporal resolution and cannot be applied for real-time monitoring of a patient during cardiac pacing. Indeed, what is required is a non-invasive technique that is easy to use for everyday applications, and would not involve extensive wiring of the subject. Such a technique must allow human-machine interface that will permit easy and fast monitoring of cerebral blood flow velocity during cardiac pacing. The said system must have high predictability of imminent compromise of cerebral blood flow in the brain of a patient. At the same time, the system must be totally non-invasive with no long-term effects on the patient.

The present invention describes a method and system for synchronization of cardiac pacing with levels of cerebral blood flow velocity in patients with cardiac pacemaker. Such an application of transcranial Doppler ultrasound as a personal carry-on device with an implanted probe has been described in U.S. Pat. No. 6,468,219B1 to Njemanze P C, however, it was a stand-alone device not hermetically in-built and functionally connected to a cardiac pacemaker. Also described is a transcranial Doppler ultrasound personal carry-on device with external probes in U.S. Pat. No. 6,390,979 to Njemanze P C but was not linked to the functionality of a cardiac pacemaker. The prior art to this day, does not describe real-time cerebral blood flow monitoring using non-invasive transcranial Doppler ultrasound during cardiac pacing as a means to maintain adequate cerebral perfusion during the different modes of pacing regimen. Similarly, prior art does not describe synchronization of cardiac pacing with brain electrical potentials such as electroencephalography or evoked potentials which could also be used as a substitute for transcranial Doppler ultrasound. Until now, no device monitors the brain function during cardiac pacing with the aim to mitigate or prevent the symptoms of cardiac pacemaker syndrome.

Cardiac pacemaker syndrome is a clinical incapacitating condition that may result in syncope and cardiovascular embarrassment. It has been demonstrated to be associated with depletion in cerebral blood flow velocity as discussed by Njemanze (1993a). There are two mechanisms that may cause syncope, the one referred to as cerebral syncope type 1 or cerebrovascular dysautoregulation syndrome and the other, cerebral syncope type 2 or vaso-vagal syncope implicating cardiac reflexes (Njemanze, 1993b). In cerebrovascular dysautoregulation syndrome, the changes in cerebral circulation precede changes in systemic arterial blood pressure and heart rate and may not implicate the cardiac reflexes as demonstrated in a heart-transplant recipient by Njemanze (1993c) and also in a heart-lung transplant recipient (Njemanze, 1992a). The detection of impending syncope could be made while measuring cerebral blood flow velocity with decrements of (−25%) from baseline values associated with premonitory signs of syncope and reduction of (−50%) resulting in syncopal episodes. The later thresholds were described as critical limits of pressure-flow relationship in the human brain by Njemanze (1992b). It therefore follows that real-time monitoring of cerebral blood flow velocity could reveal impending syncope and hence provide time for the doctor and patient to initiate mitigating measures in patients with cardiac pacemakers. The rationale of the present invention is that, it provides real-time diagnostic monitoring of cerebral circulation as well as means to initiate preventive actions during cardiac pacing. In some instances, the blood flow profile in the brain may automatically trigger the change in the cardiac pacing mode as prescribed by the doctor. The latter innovation meets a need not served by any prior art. The present invention in the embodiment termed ‘internal’ is fully hermetically integrated into the present design of the conventional cardiac pacemaker except for the monitoring transcranial Doppler ultrasound probe implanted under the skin of the temporal bone on the head. In another embodiment termed ‘external’ design of the present invention, cerebral blood flow velocity in the intracranial arteries is measured using a stand-alone externally placed transcranial Doppler ultrasound device with probe, which is telemetrically connected to the implanted cardiac pacemaker. It could be applied for diagnostic purposes for example, to determine the mental state-of-being of the patient under the different modes of cardiac pacing before permanent implantation.

In some patients, it may be necessary to implant a drug delivery system. The pump drug delivery system comprises a programmable pump, telemetrically connected to the device of the present invention and has intravenous and/or subcutaneous access from a reservoir containing drugs such as thrombolytic and neuroprotective agents. Such a pump is designed as the insulin pump that could be obtained from Medtronic, a company in Minneapolis, Minn. U.SA. In some cases of co-morbid condition with diabetes, the drugs are integrated into different compartments in the pump and administered by separate prescriptions.

The program of the device assures that the prescription information is fully accessible to the attending physician via the Internet. The continuous transcranial Doppler ultrasound monitoring, could be used interventionally for thrombolysis by emitting ultrasonic energy transmission focused on clot location to expose the clot surface to action of tissue plasminogen activator (tPA) (Alexandrov , et al. 2000). Transcranial Doppler ultrasound has been used for clinical disruption of clots or sono-thrombolysis (Zhou & Ramaswami, 2014) with tPA, and showed positive effect on early recanalization and clinical recovery rates compared to standard intra-venous tPA therapy (Molina et al. 2009).

However, in some patients due to the high risk of bleeding with anti-coagulants, the present invention could activate a mode that generates high-intensity focused ultrasound (HIFU) and histotripsy or microtripsy pulses which can effectively dissolve the blood clot without the use of thrombolytic agents according to a protocol described by Yang & Zhou, (2017).

Yet another application of the present invention is for early administration of neuroprotective agents. For example, the use of the neuroprotective agents of the class N-methyl-D-aspartate (NMDA) antagonists was based on the finding that an ischemic brain injury produces elevated levels of the excitatory neurotransmitter glutamate, which leads to excessive stimulation of the NMDA receptor as described by Lees (1997). The application of neuroprotective agents such as NMDA antagonists in acute stroke, has the advantage that they are not associated with an increased risk of hemorrhage and could therefore be administered without a screening brain imaging (CT scan or MRI). Researchers have suggested that, the administration of neuroprotective agents be commenced minutes after an infarction (Simon & Shiraishi, 1990). Acute stroke events are associated with increased discharge of microembolic clots such as in patients with atrial fibrillation (Awadh et al, 2010). The present invention provides a means to detect an acute stroke event in real-time using increased microembolic signal discharge, and to trigger the administration of neuroprotective and thrombolytic agents a few seconds thereafter. Moreover, some have suggested a combination of both thrombolytic and neuroprotective therapies (Heiss et al. 1999; Grotta, 1999). The present invention eliminates the problem of prolonged time-window from onset of event of acute stroke to administration of neuroprotective and thrombolytic agents. The clinical trial in acute stroke using neuroprotective agent Selfotel (Avantis) yielded disappointing results when administration was delayed usually within three to six hours after stroke (Davis et al., 2000). Both the neuroprotective and thrombolytic agents could be contained in an automated implanted drug injection pump, which is telemetrically connected to the present invention as a hybrid device of functionally connected transcranial Doppler ultrasound and an implanted cardiac pacemaker.

Yet another object of the present invention is the synchronization of the function of the transcranial Doppler ultrasound device with other implanted mechanical devices and external physiologic assist devices. The function of a pacemaker could be synchronized with that of the implanted transcranial Doppler ultrasound device, for example, cerebral blood flow velocity reduction during pacing could automatically trigger a functional mode of a pacemaker to maintain cerebral perfusion within a given limit.

According to an embodiment of the present invention, it incorporates synchronization of cardiac pacing with levels of cerebral blood flow velocity in a correlative relationship that maintains adequate cerebral perfusion.

According to an embodiment of the present invention, it incorporates synchronization of cardiac pacing with cerebral blood flow velocity including features of detection of micro-embolic signals in the brain vessels.

According to an embodiment of the present invention it establishes the diagnosis of factors causing the patient's symptoms such as dizziness or syncope.

According to an embodiment of the present invention, once the diagnosis of microembolic signals are made, the device alerts the doctor and the patient prompting for initiation of treatment.

According to another embodiment of the present invention, the doctor may prescribe and telemetrically transmit initiation of anticoagulant therapy by injection using automatic device from an implanted pump with venous and/or subcutaneous access.

According to yet another embodiment of the present invention, the device could detect diminishing cerebral blood flow velocity, and trigger an alert message to the patient to sit or lie down where possible to prevent orthostatic hypotension and syncope.

According to an embodiment of the present invention, the measurement of cerebral blood flow velocity is regulated in synchrony with cardiac pacing, whereby the pacing mode could be altered to achieve maintenance of cerebral blood flow autoregulation.

According to another embodiment of the present invention, an external transcranial Doppler ultrasound device with bilateral ultrasound probes on both sides of the head to assess mental performance activity in a patient with cardiac pacemaker.

According to another embodiment of the present invention, an internal transcranial Doppler ultrasound device with bilateral ultrasound probes on both sides of the head to assess mental performance activity in a patient with cardiac pacemaker.

According to another embodiment of the present invention, an external device with bilateral ultrasound probes on both sides of the head to assess mental performance activity using Artificial Intelligence machine learning methods (AI-ML) to determine the mental state-of-being in a patient with cardiac pacemaker.

According to another embodiment of the present invention, an internal device with bilateral ultrasound probes on both sides of the head to assess mental performance activity using Artificial Intelligence machine learning methods (AI-ML) to determine the mental state-of-being in a patient with cardiac pacemaker.

According to another embodiment of the present invention, an external transcranial Doppler ultrasound device with bilateral ultrasound probes on both sides of the head to assess the mental state-of-being in full awakefulness or asleep in a patient with cardiac pacemaker.

According to another embodiment of the present invention, an internal transcranial Doppler ultrasound device with bilateral ultrasound probes on both sides of the head to assess the mental state-of-being in full awakefulness or asleep in a patient with cardiac pacemaker.

According to another embodiment of the present invention, an external transcranial Doppler ultrasound device with bilateral ultrasound probes on both sides of the head to assess the mental state-of-being of the patient while processing colors to assess cognitive memory deficits in a patient with cardiac pacemaker.

According to another embodiment of the present invention, an internal transcranial Doppler ultrasound device with bilateral ultrasound probes on both sides of the head to assess the mental state-of-being of the patient while processing colors to assess cognitive memory deficits in a patient with cardiac pacemaker.

According to another embodiment of the present invention, an external transcranial Doppler ultrasound device with bilateral ultrasound probes on both sides of the head to assess the mental state-of-being of the patient while processing faces to assess cognitive memory deficits in a patient with cardiac pacemaker.

According to another embodiment of the present invention, an internal transcranial Doppler ultrasound device with bilateral ultrasound probes on both sides of the head to assess the mental state-of-being of the patient while processing faces to assess cognitive memory deficits in a patient with cardiac pacemaker.

According to another embodiment of the present invention, an external device with bilateral transcranial Doppler ultrasound probes on both sides of the head to assess the mental state-of-being of a depressive patient with a cardiac pacemaker.

According to another embodiment of the present invention, an internal device with bilateral transcranial Doppler ultrasound probes on both sides of the head to assess the mental state-of-being of a depressive patient with a cardiac pacemaker.

According to another embodiment of the present invention, an external device with bilateral transcranial Doppler ultrasound probes on both sides of the head to assess the mental state-of-being of a patient developing neurodegenerative memory deficits with a cardiac pacemaker.

According to another embodiment of the present invention, an internal device with bilateral transcranial Doppler ultrasound probes on both sides of the head to assess the mental state-of-being of a patient developing neurodegenerative memory deficits with a cardiac pacemaker.

According to another embodiment of the present invention, an internal transcranial Doppler ultrasound device with probes could be implanted on the opposite sides of the body in a patient with a pre-implanted cardiac pacemaker.

### BRIEF DESCRIPTION OF THE DRAWINGS

FIG. 1 shows a schematic block diagram that depicts one embodiment of the invention

FIG. 2 shows the implanted or ‘internal’ device of the invention.

FIG. 3 shows a brief description of the flowchart for the function of the invention.

FIG. 4 shows the ‘external’ embodiment of the present invention comprising an external transcranial Doppler ultrasound device with externally placed probes, which is telemetrically connected to a cardiac pacemaker.

FIG. 5. The schematic diagram of the present invention as ‘external’ transcranial Doppler ultrasound telemetrically connected to a cardiac pacemaker.

### DESCRIPTION OF PREFERRED EMBODIMENTS

One embodiment of the present invention would be illustrated using measurement of cerebral blood flow velocity indexed by an implanted transcranial Doppler ultrasound device with a probe telemetrically connected to a cardiac pacemaker. However, anyone skilled in the art could program the calculations of brain functional indices measured from brain electrical potentials, cerebral blood metabolism, cerebral blood flow or any other biophysiologic parameter indicative of brain cognitive function, without departing from the spirit and scope of the present invention.

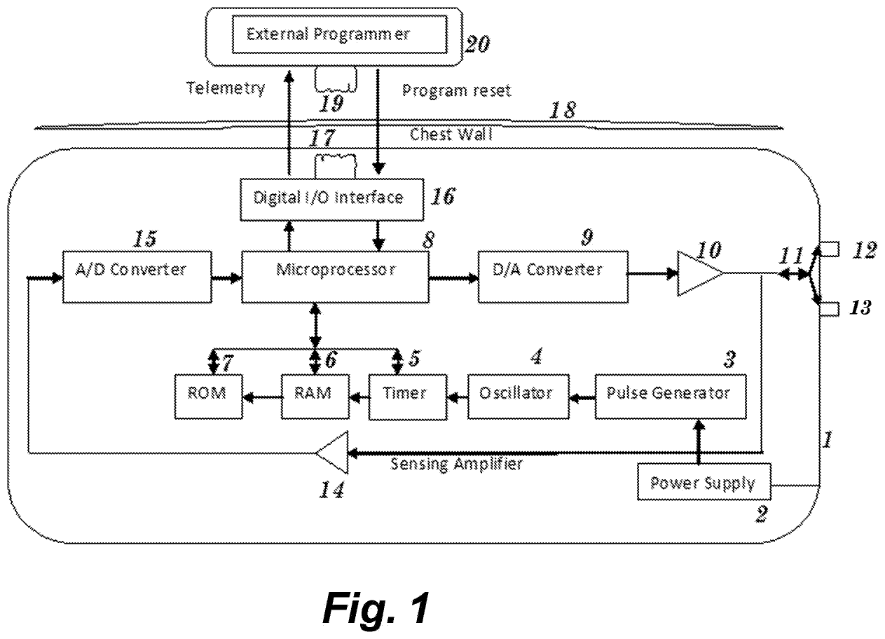

FIG. 1 shows a schematic block diagram that depicts one embodiment of the present invention.

The device is hermetically contained in a housing 1, that serves as a hybrid device comprising a cardiac pacemaker and an implantable transcranial Doppler ultrasound device. The implantable transcranial Doppler ultrasound has been described in detail according to the teachings by Njemanze P C in U.S. Pat. No. 6,468,219. The present invention generates cardiac pacing pulses, sends and receives ultrasound pulses for the implanted transcranial Doppler ultrasound probe. The present invention hermetically contains 1 electrical components that derive operating power from an internal power supply 2. The power supplies all energy for operations and electrical pulse generation as a source internal to the housing. The pulse generator 3 generates voltage for the cardiac pacemaker operation as well as the ultrasound signals and is operatively connected to the oscillator 4 and timer 5 systems. The timer 5 is operatively coupled to the memory RAM 6, ROM 7, and microprocessor 8, which are hermetically contained within the housing 1 and is communicatively coupled to the oscillator 4 and pulse generator 3. The microprocessor 8 sends signals through digital-to-analog converter 9 and amplifier 10 to connect 11 to the terminals for the electrodes 12 and ultrasound probe 13, as well as receive sensing information through an amplifier 14, and analog-to-digital converter 15. The microprocessor 8 communicates telemetrically through a digital input-output (I/O) interface 16 and antenna 17, across the chest wall 18 to the antenna 19 attached to the external programming computer 20. The microprocessor 8 has the capacity for processing the ultrasound signals required for spectrum analysis that is telemetrically communicated to the programming computer 20 for display of the blood flow waveforms and for further processing and telemetric transmission. The required information could be communicated to the patient via cell phone device through a special software application. The circuit components, leads and leadless electrodes of the cardiac pacemaker could be obtained from Medtronic a company based in Minneapolis, Minn., in the United States. The microprocessor such as that from Pentium Series could be obtained from Intel Company, of San Jose, Calif. Similarly, the circuit components and ultrasound probe for the transcranial Doppler ultrasound device could be obtained from RIMED, a company at the Industrial Park Raanana, Israel. The circuit described could be made from nano-scale components with less need for power supply, while maintaining similar functionality. There are multiple variations of the circuit and software but the illustration given here is only by way of example, and does not limit the scope of the invention.

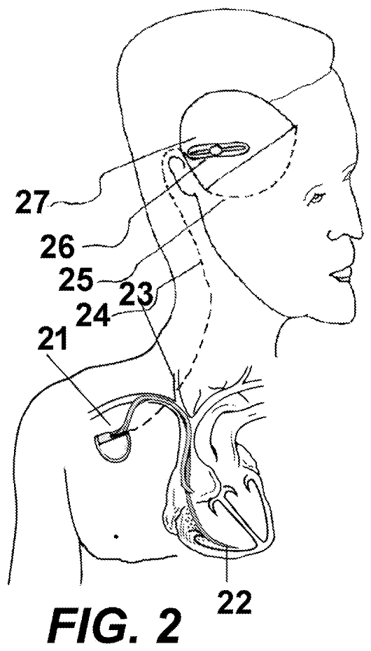

FIG. 2 shows the implanted or ‘internal’ device of the invention.

The device is implanted on the right or left side of the chest wall below the collarbone 21. The leads 22 are inserted into the jugular vein 23 and the leads guided into the correct chambers of the heart, for example, into the right ventricle and right atrium. A third cable 24 for the ultrasound probe is tunneled subcutaneously along the neck to the temporal region of the head to emerge from under the skin flap 25 that has been cut open for insertion of the ultrasound probe 26. The probe is implanted on the temporal bone window (Purkayastha & Sorond, 2012). The cable connects to the ultrasound probe 26 placed directly on the temporal bone 27. The probe emits and receives ultrasound signals focused at a particular depth in a cerebral artery for example, right middle cerebral artery. In some cases, the probe could be implanted on both sides of the temporal bones depending on indication such as when mental state-of-being needs to be monitored. The ultrasound device monitors cerebral blood flow velocity and counts microembolic signals. In some cases, there could be technical constraints such as battery life, for integration of the implanted cardiac pacemaker with the implanted transcranial Doppler ultrasound device and probe. In such cases, a separate implantable transcranial Doppler ultrasound device could be placed on the opposite side of the body with synchronization with the cardiac pacemaker. In another example, a patient with an implanted cardiac pacemaker who develops signs of pacemaker syndrome, could have a transcranial Doppler ultrasound device implanted and synchronized on the opposite side to help alleviate the symptoms.

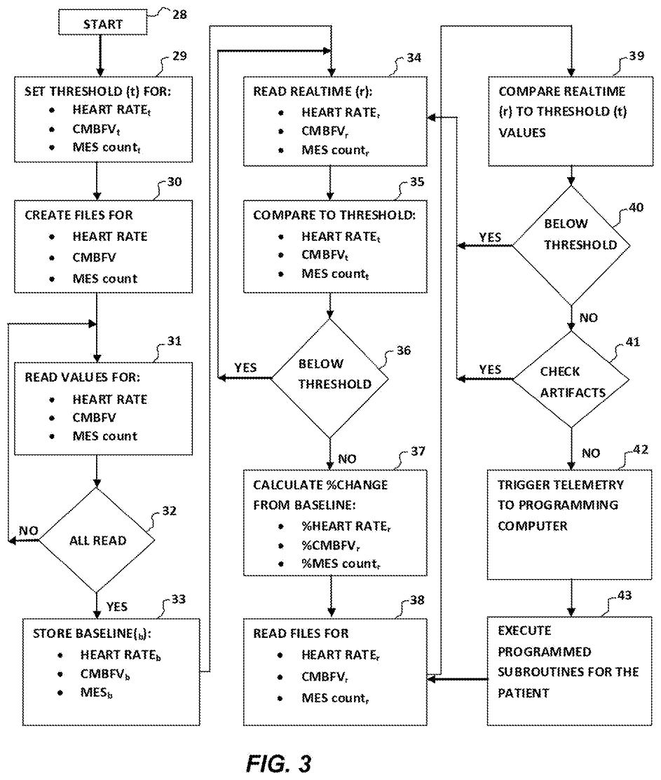

FIG. 3 shows a brief description of the flowchart for the function of the invention.

The flowchart will be illustrated by way of example. The procedure starts 28, with setting from research the predetermined threshold (t) 29 for the values of heart rate (Heart Rateₜ), cerebral mean blood flow velocity (CMBFVₜ) and microembolic signal count (MES countₜ), followed by creation of file records 30 for the parameters Heart Rate, CMBFV and MES count. The values for each parameter Heart Rate, CMBFV and MES count are read 31, if not all have been read, the previous step 31 is repeated, but if all have been read 32, the system proceeds to store the baseline values of the parameters 33 Heart Rate_{b}, CMBFV_{b}, MES count_{b}, and monitors the real-time baseline values of parameters 34 Heart Rateᵣ, CMBFVr, MES countᵣ, and compares them to the set threshold of the parameters 35 Heart Rateₜ, CMBFVₜ, MES countₜ, if below the set threshold parameters 36, then the system repeats step 34, if not, the system proceeds to calculate the percentage change of the real-time parameters 37%Heart Rateᵣ, %CMBFVr, %MES countᵣ, from baseline values. It updates the changes in real-time from the file of the parameters Heart Rateᵣ, CMBFVᵣ, MES countᵣ 38, and compares the changes to set threshold 39, and if below 40, then it continues to read the real-time values from step 34, conversely, if not, it checks for artifacts 41 using programmed sub-routines for example motion artifacts. If the artifacts are present, it repeats from step 34, but if not, it triggers telemetry to the programming computer 42 to execute programmed sub-routines specific for each patient including HIFU. The programmed sub-routines are chosen based on the clinical presentation of the patient. There are a variety of options, for example, if the system detects microembolic signals above a set threshold, artificial intelligence (AI) deep machine-learning (ML) software routine could be implemented for diagnosis and risk analysis for severe ischemic event, and the system could trigger the doctor's prescribed dosing of an injectable anti-coagulant and/or neuroprotective agent through an implanted pump, and thereafter monitor the effects on improved cerebral blood flow. The AI-ML could detect states of mental performance, sleep pattern of cerebral blood flow velocity, talking and motor-activity so as to recognize these patterns in real-time and distinguishes them from motion artifacts. The Brain Cardiac Pacemaker described below could have two probes, for example, one focused on the left middle cerebral artery (LMCA) and the other focused on the right middle cerebral artery (RMCA), and hence could monitor mental performance as described in the teachings of the U.S. Pat. No. 639,0979B1, (2002), by Njemanze P C, which could be used for the development of the AI-ML sub-routines for each individual patient. Multiple variations are possible depending on the patient's presentation, such that, each sub-routine is individualized according to the doctor's prescription.

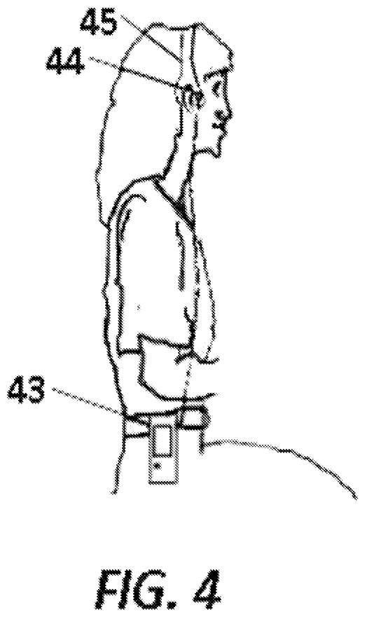

FIG. 4 shows the ‘external’ embodiment of the present invention comprising an external transcranial Doppler ultrasound device with externally placed probes, which is telemetrically connected to a cardiac pacemaker.

The design of the external unit of the present invention eliminates the need for revision of functioning implanted cardiac pacemakers. Rather the pacing mode of the cardiac pacemaker could be adjusted based on the mental state-of-being of the patient after evaluation with the external unit of the present invention. A choice could be made on the best mode of pacing to optimize mental performance assessed clinically or according to the teachings of U.S. Pat. No. 6,390,979 to Njemanze, 2002.

In some patients with cardiac pacemakers, the doctor may choose to synchronize the function with an externally mounted transcranial Doppler ultrasound device 43 with ultrasound head probes 44 placed on the temporal bone mounted on a probe hanger 45, from both sides of the head. The transcranial Doppler ultrasound device synchronizes blood flow velocity measurements with cardiac pacing. The transcranial Doppler ultrasound device is telemetrically communicating with the microprocessor of the cardiac pacemaker to achieve the same functionality as described above in FIG. 3. The design of the external transcranial probe for long-time monitoring could be done in a number of ways for example, as described by in U.S. Pat. No 6,547,737B2 by Njemanze P C. In the clinical setting, the external transcranial Doppler ultrasound is used to evaluate if the symptoms of the patient could be relieved by monitoring the cerebral blood flow velocity and synchronizing the function to different modes of cardiac pacing. The external Brain Cardiac Pacemaker is used for development of AI-ML sub-routines for each patient in the training phase. Once the clinical goals are achieved for the patient, the Brain Cardiac Pacemaker with the permanent telemetric transcranial Doppler ultrasound device with probe could be implanted.

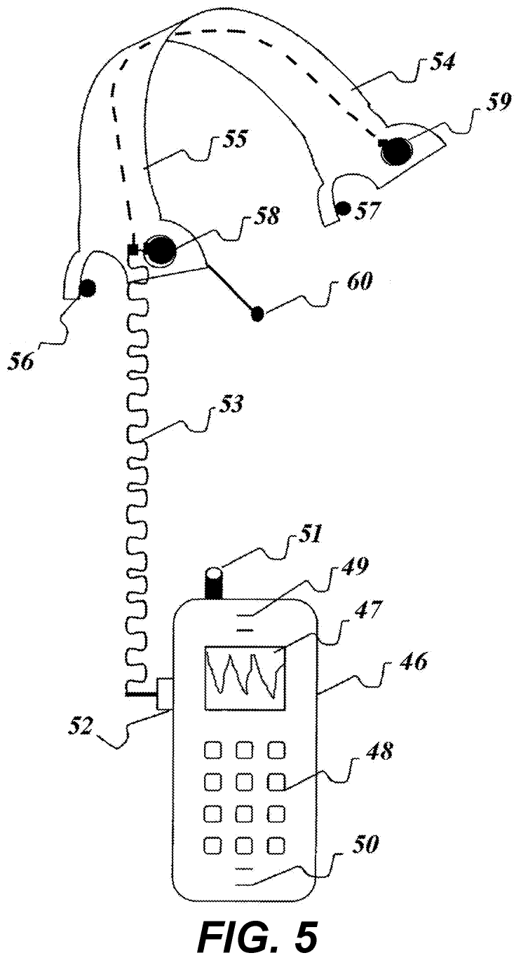

FIG. 5. The schematic diagram of the present invention as ‘external’ transcranial Doppler ultrasound telemetrically connected to an implanted cardiac pacemaker.

FIG. 5. shows the device that has the form of a cellular telephone device 46 with the usual features of an LCD display 47, an input keypad 48, a loudspeaker 49 and a microphone 50. An aerial receiver 51 for radio frequency transmission and reception of signals in addition to locating the user's position using global positioning system (GPS). The device has means for telemetric connection including radio frequency, Bluetooth and others. The present invention has a headset connected to an adapter terminal 52 through a cable 53 to the headset with left 54 and right 55 sides placed on the left temporal and right temporal bones, respectively. Each side of the headset has an ear piece on the right 56 and left 57 for audio communication to the patient. There are transcranial Doppler ultrasound probes on the right 58 and left 59 temporal bones contained in the probe housing and focused for measurement of cerebral blood flow velocities in intracranial arteries. The synchronous bilateral probes have artificial intelligent (AI) automated angulation for insonation of cerebral arteries according to the teachings of Njemanze in U.S. Pat. No. 6,547,737. If the patient speaks, the system monitors the audio signals through a microphone 60. The present invention was designed as a portable device to monitor cerebral blood flow velocity which is telemetrically synchronized with a cardiac pacemaker. The cellular phone packaging parts could be obtained from Nokia a company based in Espoo, Finland. The microprocessor such as that from Pentium Series could be obtained from Intel Company, of San Jose, Calif. by way of example. This present invention is a composite device comprising function of a microcomputer for the transcranial Doppler ultrasound, a cellular phone telemetrically connected to the functionality of an implanted cardiac pacemaker. It could be applied in clinical situations when cerebral blood flow velocity needs to be monitored and telemetrically synchronized to another biophysiologic parameter.

I claim:

1. A system for cardiac pacing that involves monitoring of cerebral blood flow in a patient with cardiac pacemaker.

2. A system of claim 1 wherein the real-time monitoring of cerebral blood flow velocity using a transcranial Doppler ultrasound instrument is functionally integrated into an implanted cardiac pacemaker in a patient.

3. A system of claim 1 wherein the real-time monitoring of cerebral blood flow velocity uses an implanted transcranial Doppler ultrasound probe placed on one or both sides of the temporal bone in a patient with cardiac pacemaker.

4. A system of claim 1 wherein the real-time monitoring of cerebral blood flow velocity is used to select the cardiac pacing mode for best mental performance.

5. A system of claim 1 wherein the real-time monitoring of cerebral blood flow velocity is synchronized with the function of the cardiac pacemaker and is telemetrically connected with the cell phone and a programming computer.

6. A system of claim 1 wherein the real-time monitoring of cerebral blood flow velocity uses an implanted transcranial Doppler ultrasound probe during cardiac pacing to detect the presence of microemboli in the brain circulation.

7. A system of claim 1 wherein the real-time monitoring of cerebral blood flow velocity uses an implanted transcranial Doppler ultrasound probe during cardiac pacing to detect the presence of cerebral microembolic signals and is programmed to administer thrombolytic and neuroprotective medication including through an automatic implanted drug injection pump.

8. A system of claim 1 wherein the real-time monitoring of cerebral blood flow velocity uses implanted ultrasound probes on both sides of the temporal bones synchronized with cardiac pacing to determine the mental state-of-being in a patient and to alter the cardiac pacing mode to enhance cerebral perfusion.

9. A system of claim 1 wherein the real-time monitoring of cerebral blood flow velocity uses implanted ultrasound probes on both sides of the temporal bones synchronized with cardiac pacing to determine the mental state-of-being in a patient with depression, cognitive decline or memory deficits with the aim to alter the cardiac pacing mode to enhance cerebral perfusion.

10. A system of claim 1 wherein the real-time monitoring of cerebral perfusion is indexed using brain electrical potentials that are synchronized with cardiac pacing to determine the mental-state of being in a patient with implanted cardiac pacemaker and to alter the pacing mode to enhance mental performance.

11. A system for cardiac pacing that hermetically contains the circuits for an implanted cardiac pacemaker integrated and synchronized with that of an implanted transcranial Doppler ultrasound device for measurement of cerebral blood flow velocity said system comprising: pulse generator, microprocessor, power supply system, oscillator, timer, memory digital/analog converters, amplifiers, radio-frequency transmitter for telemetric transmission to an external programming computer, with connections to pacing leads or leadless electrodes and implanted transcranial Doppler ultrasound probe.

12. A system of claim 11 wherein the real-time monitoring of cerebral blood flow velocity uses a synchronized but separately implanted transcranial Doppler ultrasound device with probe placed on the temporal bone on the opposite side of a patient with cardiac pacemaker.

13. A system of claim 11 wherein the real-time monitoring of cerebral blood flow velocity is synchronized with cardiac pacing, said method to select the appropriate pacing mode of the implanted cardiac pacemaker using artificial intelligence.

14. A system of claim 11 wherein the real-time monitoring of cerebral blood flow velocity indexed by a transcranial Doppler ultrasound device with probe in a patient with cardiac pacemaker could be made from nano-scale circuit components with lesser need for power supply.

15. A system of claim 11 wherein the real-time monitoring of cerebral blood flow velocity is used to determine microembolic signals, said method to trigger delivery of anticoagulant and neuroprotective agents and sonothrombolysis.

16. A system of claim 11 wherein the real-time monitoring of cerebral blood flow velocity is used to select the cardiac pacing mode that maintains cerebral perfusion for optimal mental performance.

17. A system for non-invasive monitoring of cerebral blood flow velocity using an external transcranial Doppler ultrasound device with external probes, that is telemetrically connected to a cardiac pacemaker implanted in a patient, said system comprising: pulse generator, microprocessor, power supply system, oscillator, timer, memory digital/analog converters, amplifiers, radio-frequency transmitter for telemetric transmission to the cardiac pacemaker and external programming computer.

18. A system of claim 17 wherein the real-time monitoring of cerebral blood flow velocity is used to determine the mental state-of-being by applying artificial intelligence in a patient with an implanted cardiac pacemaker.

19. A system of claim 17 wherein the real-time monitoring of cerebral blood flow velocity uses an external transcranial Doppler ultrasound device and external ultrasound probes placed on the temporal bones, that is telemetrically connected to the cardiac pacemaker implanted in a patient, said method used to select the pacing mode that maintains adequate cerebral perfusion, to detect microembolic signals and to trigger injection of thrombolytic and neuroprotective agents.

20. A system of claim 17 wherein the real-time monitoring of cerebral blood flow velocity uses an external transcranial Doppler ultrasound device and probes placed on the temporal bones, that is telemetrically connected to a cardiac pacemaker implanted in a patient, said method is connected to a cell phone for communication with the patient and doctor.
